(19) United States
(12) Patent Application Publication
(10) Pub. No.: US 2024/0207564 A1
(43) Pub. Date: Jun. 27, 2024

(54) DIFFUSED EXHALATION WATER MANAGEMENT

(71) Applicant: KONINKLIJKE PHILIPS N.V., EINDHOVEN (NL)

(72) Inventors: ALEXANDER ENZMAN, PITTSBURGH, PA (US); ANNA DZURICKY, ERIE, PA (US); LAUREN PATRICIA CHODKOWSKI, PITTSBURGH, PA (US)

(21) Appl. No.: 18/544,607

(22) Filed: Dec. 19, 2023

Related U.S. Application Data
(60) Provisional application No. 63/434,496, filed on Dec. 22, 2022.

(51) Int. Cl. A61M 16/08; A61M 16/06

(52) U.S. Cl. CPC A61M 16/0883; A61M 16/0616; A61M 16/0875

(57) ABSTRACT

An exhaust arrangement for exhausting gases from a passage defined within a portion of a patient circuit used in providing a flow of a breathing gas to an airway of a patient, the exhaust arrangement comprising: a body defining a number of exhaust ports passing therethrough, each exhaust port having an inlet and an outlet and sized and configured to provide for the passage of gasses therethrough; and a raised portion positioned about the number of exhaust ports and extending from the body, wherein the raised portion is sized and configured to extend into the passage of the portion of the patient circuit from an inner surface of the portion of the patient circuit immediately adjacent the exhaust arrangement.

### CROSS-REFERENCE TO RELATED APPLICATIONS

This patent application claims the priority benefit under 35 U.S.C. § 119(e) of U.S. Provisional Application No. 63/434,496, filed on Dec. 22, 2022, the contents of which are herein incorporated by reference.

### BACKGROUND OF THE INVENTION

### 1. Field of the Invention

The present invention relates to arrangements for communicating a flow of a treatment gas to the airway of a patient and, more particularly, to exhaust arrangements having one or more features for inhibiting the passage of water therethrough.

### 2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies involve the placement of a patient interface device including a mask component on the face of a patient. The mask component may be, without limitation, a nasal mask that covers the patient's nose, a nasal cushion having nasal prongs that are received within the patient's nares, a nasal/oral mask that covers the nose and mouth, or a full face mask that covers the patient's face. The patient interface device interfaces a ventilator or pressure support device with the airway of the patient, so that a flow of breathing gas can be delivered from a pressure/flow generating device to the airway of the patient.

In addition to providing the flow of breathing gas to the airway of the patient, such arrangements must also account for and provide for the exhaust of exhalation gases produced by the patient from the system. In the current CPAP market, arrangements for exhausting exhalation gases of the patient are commonly found in the elbows or on the front of a faceplate or similar element of a mask. Such arrangements are commonly oriented in one direction so if a patient were to lay with the air flow jetting towards the pillow a loud noise will be created, disturbing the patient/user's sleep as well as that of a bed partner of the patient. Additionally, such concentrated jet can commonly end up aimed at the aforementioned bed partner which is likewise undesirable.

In attempting to address the aforementioned problems, exhaust arrangements utilizing a felt or similar material positioned across the exhaust ports have been employed. While such arrangements tend to do a good job diffusing the exhaust, thus improving upon the aforementioned problems, such diffused arrangements present a new problem in handling water from the system. Water that condenses within a CPAP mask that has diffused exhalation, can have difficulty expelling from the mask's exhalation. This may result from a wet diffuser partially or entirely blocking the exit path, and/or capillary action building up in the exhalation features that is not blown out by the CPAP pressure or forces of exhalation by the patient. When the diffuser felt material contacts water, it significantly reduces the flow that can pass through the membrane and water builds in the material. There is therefore a need to provide improved exhaust arrangements for use with CPAP masks and related components.

### SUMMARY OF THE INVENTION

Accordingly, it is an object of the present invention to provide solutions which address shortcomings of conventional arrangements. As one aspect of the present invention an exhaust arrangement for exhausting gases from a passage defined within a portion of a patient circuit used in providing a flow of a breathing gas to the airway of a patient is provided. The exhaust arrangement comprises: a body defining a number of exhaust ports passing therethrough, each exhaust port having an inlet and an outlet and sized and configured to provide for the passage of gasses therethrough; and a raised portion positioned about the number of exhaust ports and extending from the body. The raised portion is sized and configured to extend into the passage of the portion of the patient circuit from an inner surface of the portion of the patient circuit immediately adjacent the exhaust arrangement.

The portion of the patient circuit may comprise a patient interface; and the passage of the portion may comprise a main cavity of the patient interface. The portion of the patient circuit may comprise a supply conduit; and the passage of the portion may comprise a supply passage of the supply conduit. The raised portion may completely encircle the exhaust port. The number of exhaust ports may comprise only a single exhaust port. The number of exhaust ports may comprise a plurality of exhaust ports. The body of the exhaust arrangement may be structured to be positioned in an aperture defined in the portion of the patient circuit and coupled to the portion of the patient circuit. The body of the exhaust arrangement may be formed integrally as a portion of the portion of the patient circuit. The raised portion may comprise a flange portion extending radially outward from the number of exhaust ports, wherein the flange portion is sized and configured to overhang a region of, and be spaced a predetermined distance from, the inner surface of the portion of the patient circuit immediately adjacent the exhaust arrangement.

As another aspect of the present invention, a patient circuit for use in providing a flow of breathing gas to an airway of a patient from a pressure generating device is provided. The patient circuit comprises: a delivery conduit having a first end structured to be coupled to the pressure generating device and a second end opposite the first end, the delivery conduit structured to convey the flow of breathing gas from the first end to the second end; a patient interface device coupled to the second end of the delivery conduit and structured to engage about the airway of the patient and convey the flow of breathing gas from the second end of the delivery conduit to the airway of the patient; and an exhaust arrangement for exhausting gases from a passage defined within one of the delivery conduit or the patient interface device, the exhaust arrangement comprising: a body defining a number of exhaust ports passing therethrough, each exhaust port having an inlet and an outlet and sized and configured to provide for the passage of gasses therethrough; and a raised portion positioned about the number of exhaust ports and extending from the body, wherein the raised portion is sized and configured to extend into the passage of the one of the delivery conduit or the patient interface device from an inner surface thereof immediately adjacent the exhaust arrangement.

The exhaust arrangement may be provided as portion of the patient interface; and the passage may comprise a main cavity of the patient interface. The exhaust arrangement may be provided as a portion of the supply conduit; and the passage may comprise a supply passage of the supply conduit. The raised portion may completely encircle the exhaust port. The body of the exhaust arrangement may be formed integrally as a portion of the one of the delivery conduit or the patient interface device. The raised portion may comprise a flange portion extending radially outward from the number of exhaust ports, wherein the flange portion is sized and configured to overhang a region of, and be spaced a predetermined distance from, the inner surface immediately adjacent the exhaust arrangement.

These and other objects, features, and characteristics of the disclosed concept, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a partially schematic view of a respiratory interface system in accordance with one example embodiment of the present invention;

FIG. 2 is an elevation front view of the patient interface of the respiratory interface system of FIG. 1;

FIG. 3 is a sectional view of the patient interface of FIG. 2 taken along 3-3 of FIG. 2 showing an example exhaust arrangement in accordance with an example embodiment of the present invention;

FIG. 4 is an enlarged view of the portion of the sectional view of FIG. 3 as indicated in FIG. 3;

FIG. 4A is a view of the exhaust arrangement of FIGS. 3 and 4 as viewed from the viewpoint shown by the arrow indicated in FIG. 4;

FIG. 5-7 are enlarged views similar to that of FIG. 4 but instead showing sectional views of other example exhaust arrangements in accordance other example embodiments of the present invention;

FIG. 8 is a partially schematic view of another respiratory interface system in accordance with another example embodiment of the present invention; and

FIGS. 9 and 10 are sectional views of a portion of a delivery conduit of the system of FIG. 8 showing details of exhaust arrangements in accordance with further embodiments of the present invention.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As used herein, the statement that two or more parts or components “engage” one another shall means that the parts exert a force against one another either directly (i.e., “directly engage”) or through one or more intermediate parts or components. As used herein, the word “unitary” means a component is created as a single piece or unit. That is, a component that includes pieces that are created separately and then coupled together as a unit is not a “unitary” component or body. As used herein, the term “number” shall mean one or an integer greater than one (i.e., a plurality).

Referring to FIG. 1, a respiratory interface system 2 for use in providing a regimen of respiratory therapy to a patient (not shown) according to one exemplary embodiment of the present invention is shown. Respiratory interface system 2 includes a pressure generating device 4 (shown schematically), a delivery conduit 6 (shown partially schematically) coupled between pressure generating device and a tubing assembly 8, and a patient interface 10 coupled to tubing assembly 8. Pressure generating device 4 is structured to generate a flow of positive pressure breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, PA), and auto-titration pressure support devices. Delivery conduit 6 is structured to communicate the flow of breathing gas from pressure generating device 4 to patient interface device 10 through tubing assembly 8 (the flow of breathing gas enters tubing assembly 8 at the top of the head of a patient when tubing assembly 8 and patient interface device 10 are positioned on the head of the patient). Delivery conduit 6, tubing assembly 8 and patient interface device 10 are often collectively referred to as a patient circuit.

Continuing to refer to FIG. 1, tubing assembly 8 includes a number of tubular portions 12 which each extend from a common manifold portion 14 to a respective distal end 16. Distal end 16 of each tubular portion 12 is coupled to patient interface 10. In exemplary embodiments, tubing assembly 8 is made from plastic and/or silicone and may be formed as a single unitary member or alternately may be formed from a number of separately formed components that are then coupled together via a suitable process. Tubing assembly 8 and/or patient interface 10 may also be formed from other suitable materials (e.g., fabric) without varying from the scope of the present invention.

Manifold portion 14 is structured to be coupled to delivery conduit 6 and in the example of FIG. 1 is shown coupled to an elbow 18 of deliver conduit 6. When tubing assembly 8 is disposed on the head of a patient, manifold portion 14 is disposed generally at the top of the head of the patient and tubular portions 12 each extend generally downward from manifold portion 14 to patient interface device 10. In the exemplary embodiment shown in FIG. 1, tubing assembly 8 includes two tubular portions 12, also referred to herein as left side arm 20 and right side arm 22, which each have a generally non-circular cross-section. That is, each tubular portion 12 is not substantially circular. In another exemplary embodiment, not shown, tubing assembly 8 includes a single tubular portion 12 that extends centrally, i.e., from manifold portion 14 generally over the patient's forehead and nose, to patient interface 10.

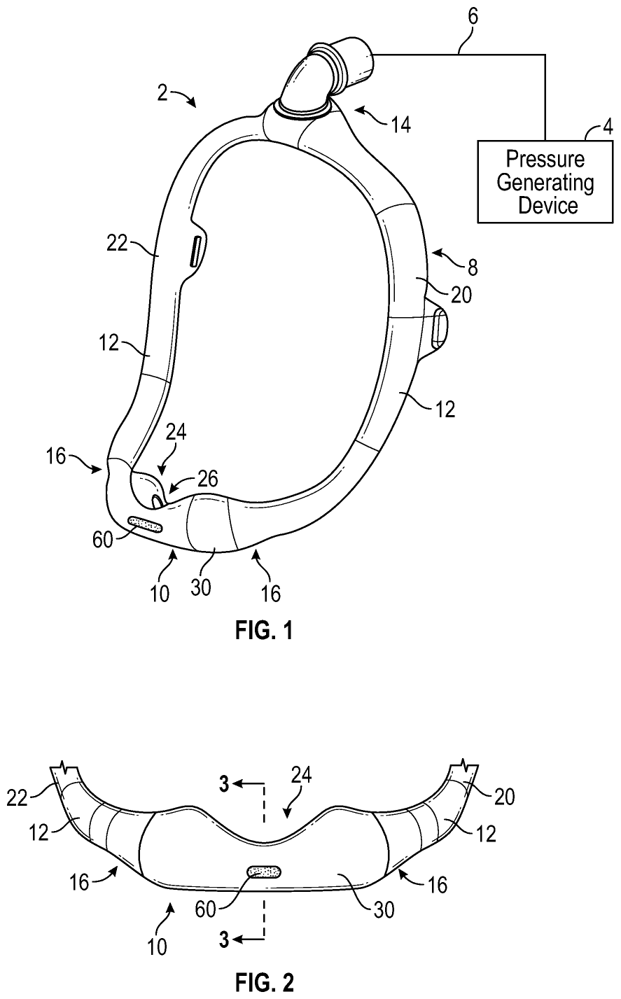

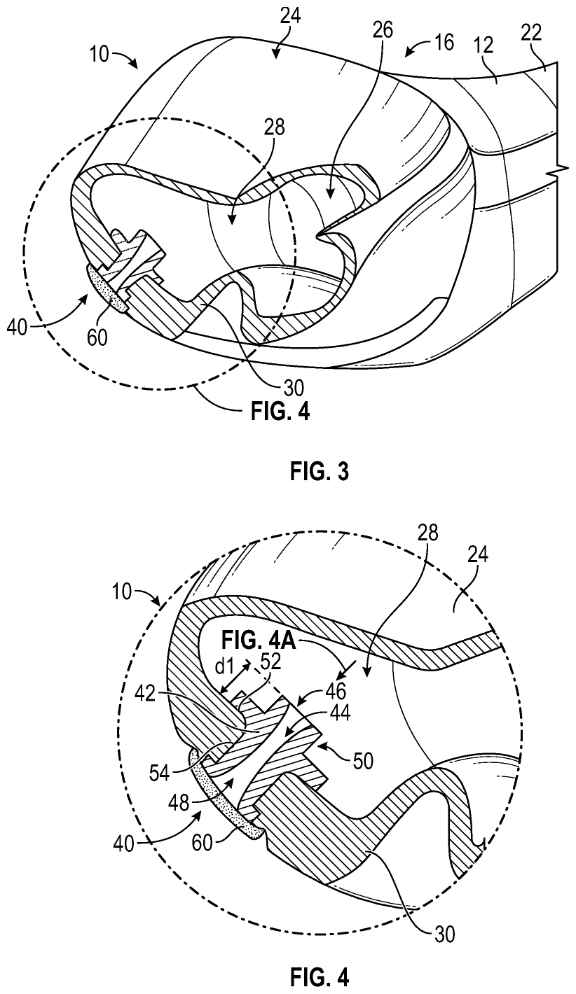

Continuing to refer to FIG. 1, as well as to FIGS. 2 and 3, patient interface 10 includes a sealing arrangement 24 that is structured to seal generally about an airway of a patient. In an exemplary embodiment, such as illustrated in FIG. 1, sealing arrangement 24 is a nasal cushion made of a soft, flexible material, such as, without limitation, silicone, an appropriately soft thermoplastic elastomer, a closed-cell foam, or any other suitable material or combination of such materials, however, it is to be appreciated that any type of sealing arrangement, such as a nasal/oral mask, a nasal pillow or a full face mask, which facilitates the delivery of the flow of breathing gas to the airway of a patient, may be used as sealing arrangement 24 while remaining within the scope of the present invention.

Sealing arrangement 24 is positioned about a patient aperture 26 which provides access to a main cavity 28 (FIG. 3) defined within a body 30 of patient interface 10. Main cavity 28 is structured to receive the flow of breathing gas produced by pressure generating device 4 (and conveyed to main cavity 28 via delivery conduit 6 and tubular portions 12 of tubing assembly 8) which is then conveyed to the airway of a patient through patient aperture 26. In the example embodiment illustrated in FIG. 1, main cavity 28 receives the flow of breathing gas produced by pressure generating device 4 via a pair of inlet ports (not numbered) positioned at either end of patient interface 10 that are each coupled to a respective distal end 16 of left and right-side arms 20, 22.

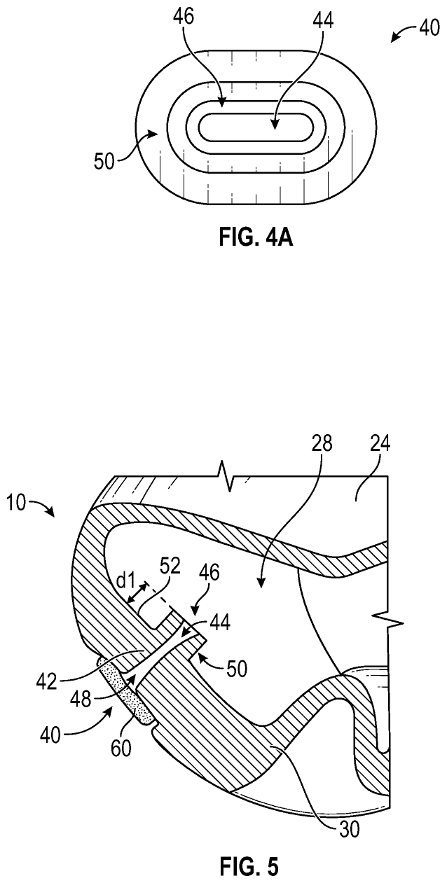

Patient interface 10 further includes an exhaust arrangement 40 in accordance with one example embodiment of the present invention that provides for the exhausting of gases from patient interface 10, and more particularly from main cavity 28 of patient interface 10. Referring to FIG. 3 and the enlarged portion thereof shown in FIG. 4, exhaust arrangement 40 includes a body 42 defining a number of exhaust ports 44 passing therethrough. Each exhaust port includes an inlet 46 and an outlet 48 and is sized and configured to provide for the passage of gasses therethrough. In the example shown in FIG. 4, body 42 defines only a single exhaust port 44 having a generally elliptical cross-section (e.g., see FIG. 4A), however, it is to be appreciated that one or more of quantity and or cross-sectional shape of the exhaust port(s) 44 may be varied without varying from the scope of the present invention.

Exhaust arrangement 40 further includes a raised portion 50 positioned about the number of exhaust ports 44. Raised portion 50 is sized and configured to extend into main cavity 28 of patient interface 10 a first predetermined distance d1 beyond an inner surface 52 of patient interface 10 immediately adjacent exhaust arrangement 40. As shown in FIG. 4A, raised portion 50 completely encircles exhaust port 44, however, it is to be appreciated that in certain arrangements raised portion 50 may encircle only a portion of exhaust port 44 without varying from the scope of the present invention.

Body 42 of the example exhaust port arrangement 40 shown in FIGS. 3 and 4 is structured so as to be positioned and coupled in an aperture 54 defined in body 30 of patient interface 10. Such coupling may be of a generally permanent manner (e.g., without limitation, via adhesive, sonic welding, etc.) or via a selective manner such that exhaust port arrangement may be selectively uncoupled and removed (e.g., without limitation, for cleaning, swapping for new/other sizes/other arrangements, etc.). In contrast, as shown in the example embodiment of FIG. 5, exhaust arrangement 40 may be formed as an integral portion of patient interface 10 (i.e., body 42 of exhaust arrangement 40 formed as a unitary portion of body 30 of patient interface 10). In either case, exhaust arrangement 40 may include a diffuser element 60 positioned (via any suitable coupling arrangement) covering all, or a portion, of outlet 48 of exhaust port 44.

Diffuser element 60 may be composed of a porous or other suitable material that can effectively absorb airflow, e.g., without limitation, felt, fabric(s), spacer fabric(s), foam(s), mesh(es), filter material(s), etc. and thus be suitable for diffusing a flow of gases passing through exhaust port 44. It is to be appreciated that in either of such example arrangements that raised portion 50 serves to inhibit water present on the inner surface(s) of patient interface 10 from entering exhaust port 44 and saturating diffuser element 60.

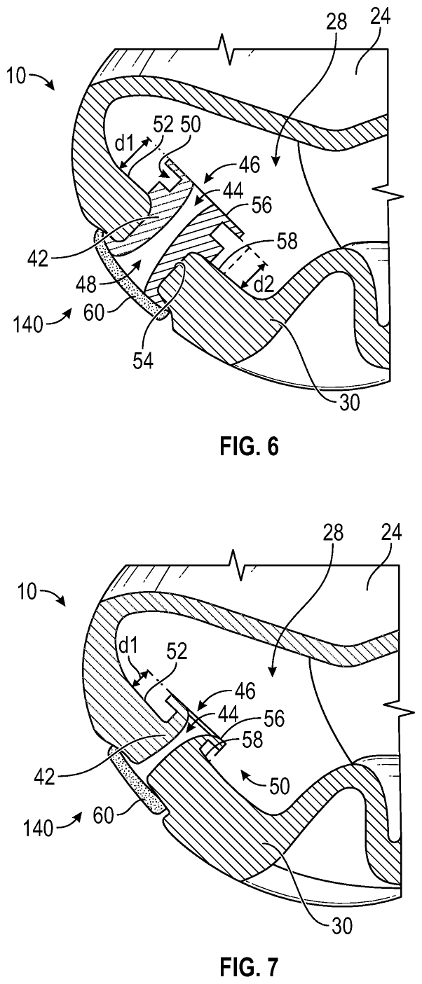

Referring now to FIGS. 6 and 7, other example embodiments of an exhaust arrangement 140 similar to exhaust arrangement 40 of FIGS. 4 and 5 is shown. Exhaust arrangement 140 includes the elements of exhaust arrangement 40 except raised portion 50 thereof further includes a flange portion 56 extending radially outward from the number of exhaust ports 44. Flange portion 56 is sized and configured to overhang a region (shown generally at 58 in FIG. 6) of, and be spaced a predetermined distance d2 from, inner surface 52 of patient interface 10 immediately adjacent exhaust arrangement 140 thus forming a cavity adjacent raised portion 50. Such flange portion 56 serves as an even stronger inhibitor against water present on the inner surface(s) of patient interface 10 from entering exhaust port 44 and saturating diffuser element 60.

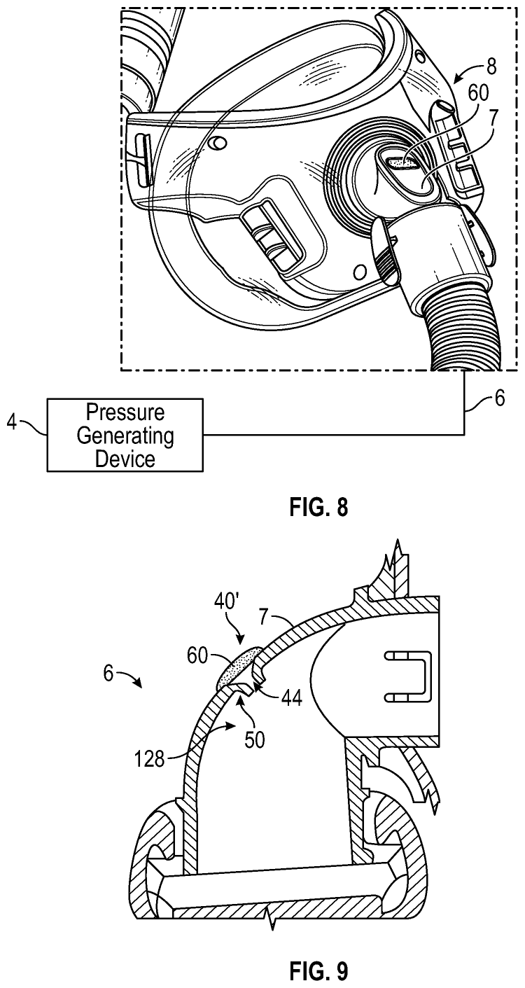

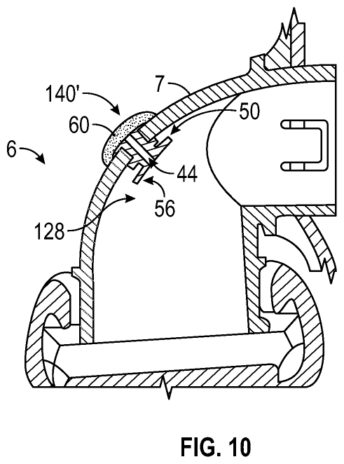

While the foregoing examples have shown example exhaust arrangements positioned for handling gases exhausted directly from the main cavity of a patient interface portion of a patient circuit, it is to be appreciated that embodiments of the present invention may also be employed for exhausting gases elsewhere along the general supply passage defined by components of the patient circuit without varying from the scope of the present invention. For example, as shown in FIGS. 8-10, exhaust arrangements such as described herein may be employed in supply conduit 6 (shown in an elbow member 7 provided as a portion of supply conduit 6) for exhausting gases from a supply passage 128 (i.e., similar to cavity 28) defined in supply conduit. For example, FIG. 9 illustrates an example exhaust arrangement 40′ similar to exhaust arrangement 40 shown in FIG. 5, while FIG. 10 illustrates an example exhaust arrangement 140′ similar to exhaust arrangement 140 shown in FIG. 6.

From the examples and accompanying descriptions it is thus to be appreciated that embodiments of the present invention provide arrangements for exhausting gases from passages defined within portions of patient circuits used in providing a flow of a breathing gas to the airway of a patient and, more particularly, arrangements that not only provide for the exhausting of gases but also that inhibit/prohibit water present on nearby surfaces from exiting and causing negative effects on diffuser material(s) present at such exhaust point.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. An exhaust arrangement for exhausting gases from a passage defined within a portion of a patient circuit used in providing a flow of a breathing gas to an airway of a patient, the exhaust arrangement comprising:
a body defining a number of exhaust ports passing therethrough, each exhaust port having an inlet and an outlet and sized and configured to provide for the passage of gasses therethrough; and
a raised portion positioned about the number of exhaust ports and extending from the body,
wherein the raised portion is sized and configured to extend into the passage of the portion of the patient circuit from an inner surface of the portion of the patient circuit immediately adjacent the exhaust arrangement.

2. The exhaust arrangement of claim 1, wherein:
the portion of the patient circuit comprises a patient interface; and
the passage of the portion comprises a main cavity of the patient interface.

3. The exhaust arrangement of claim 1, wherein:
the portion of the patient circuit comprises a supply conduit; and
the passage of the portion comprises a supply passage of the supply conduit.

4. The exhaust arrangement of claim 1, wherein the raised portion completely encircles the exhaust port.

5. The exhaust arrangement of claim 1, wherein the number of exhaust ports comprises only a single exhaust port.

6. The exhaust arrangement of claim 1, wherein the number of exhaust ports comprises a plurality of exhaust ports.

7. The exhaust arrangement of claim 1, wherein the body of the exhaust arrangement is structured to be positioned in an aperture defined in the portion of the patient circuit and coupled to the portion of the patient circuit.

8. The exhaust arrangement of claim 1, wherein the body of the exhaust arrangement is formed integrally as a portion of the portion of the patient circuit.

9. The exhaust arrangement of claim 1, wherein the raised portion comprises a flange portion extending radially outward from the number of exhaust ports, wherein the flange portion is sized and configured to overhang a region of, and be spaced a predetermined distance (d2) from, the inner surface of the portion of the patient circuit immediately adjacent the exhaust arrangement.

10. A patient circuit for use in providing a flow of breathing gas to an airway of a patient from a pressure generating device, the patient circuit comprising:
a delivery conduit having a first end structured to be coupled to the pressure generating device and a second end opposite the first end, the delivery conduit structured to convey the flow of breathing gas from the first end to the second end;
a patient interface device coupled to the second end of the delivery conduit and structured to engage about the airway of the patient and convey the flow of breathing gas from the second end of the delivery conduit to the airway of the patient; and
an exhaust arrangement for exhausting gases from a passage defined within one of the delivery conduit or the patient interface device, the exhaust arrangement comprising:
a body defining a number of exhaust ports passing therethrough, each exhaust port having an inlet and an outlet and sized and configured to provide for the passage of gasses therethrough; and
a raised portion positioned about the number of exhaust ports and extending from the body,
wherein the raised portion is sized and configured to extend into the passage of the one of the delivery conduit or the patient interface device from an inner surface thereof immediately adjacent the exhaust arrangement.

11. The patient circuit of claim 10, wherein:
the exhaust arrangement is provided as portion of the patient interface; and
the passage comprises a main cavity of the patient interface.

12. The patient circuit of claim 10, wherein:
the exhaust arrangement is provided as a portion of the supply conduit; and
the passage comprises a supply passage of the supply conduit.

13. The patient circuit of claim 10, wherein the raised portion completely encircles the exhaust port.

14. The patient circuit of claim 10, wherein the body of the exhaust arrangement is formed integrally as a portion of the one of the delivery conduit or the patient interface device.

15. The patient circuit of claim 10, wherein the raised portion comprises a flange portion extending radially outward from the number of exhaust ports, wherein the flange portion is sized and configured to overhang a region of, and be spaced a predetermined distance from, the inner surface immediately adjacent the exhaust arrangement.
